(19) United States
(12) Patent Application Publication
(10) Pub. No.: US 2015/0073531 A1
(43) Pub. Date: Mar. 12, 2015

(54) DRUG ELUTING DEPOT STENT WITH ENHANCED FATIGUE LIFE

(71) Applicant: NATIONAL TAIWAN UNIVERSITY, TAIPEI CITY (TW)

(72) Inventors: HAO-MING HSIAO, TAIPEI CITY (TW); LI-WEI WU, TAIPEI CITY (TW); MING-TING YIN, TAIPEI CITY (TW); CHENG-HAN LIN, TAIPEI CITY (TW)

(21) Appl. No.: 14/105,736

(22) Filed: Dec. 13, 2013

(30) Foreign Application Priority Data
Sep. 11, 2013 (TW) 102132783

(51) Int. Cl. A61F 2/89; A61L 31/16

(52) U.S. Cl. CPC A61F 2/89; A61L 31/16
USPC 623/1.16

(57) ABSTRACT

A drug eluting depot stent is provided. The stent has two free ends and a tubular body connected therebetween. The tubular body includes a series of rings having undulating structures. Each of the undulating structure has a bar arm and a crown connected thereto. The bar arm has a first end portion, a second end portion and a mid-section defined therebetween. The bar arm has opened regions defined at the first end portion, the mid-section, and the second end portion, respectively. The opening ratio of the mid-section is larger than that of the two end portions. With the aforementioned structure, the stress concentration at the crown region can be re-distributed towards the bar arm, and thus effectively prolonging the fatigue life of the stent.

### BACKGROUND OF THE INVENTION

1. Field of the Invention

The instant disclosure relates to a drug eluting depot stent for implant into human blood vessels.

2. Description of Related Art

Typically, stents are clinically used for treatment of vascular diseases, specifically to treat stenosis or vessel occlusion, to be implanted into narrowed or diseased blood vessels via various catheter techniques.

In principle, stent can provide support to the periphery of the blood vessels such that the blood vessels will not collapse. However, in reality, after the stent is implanted in the blood vessels, cell proliferation on the inner walls of the blood vessels may occur, which may clog the blood vessels once again.

In order to prevent the blood vessels from restenosis, current drug eluting stents in the market attempts to elute drugs which inhibit cell proliferation and reduce the chances of further re-clogging. However, the structural strength and the service life of the drug eluting depot stents are typically lower in comparison with the conventional drug eluting stents without drug reservoirs, which limits their respective usage.

To address the above issues, the inventor strives via associated experience and research to present the instant disclosure, which can effectively improve the limitation described above.

### SUMMARY OF THE INVENTION

The object of the instant disclosure is to provide a drug eluting depot stent which has openings of various cross-sectional shapes and sizes for drug retention, and more uniform stresses across the stent in order to prolong the service life of the stent.

In order to achieve the aforementioned objects, according to an embodiment of the instant disclosure, a drug eluting depot stent is provided which includes two free ends and a tubular body. The tubular body has a longitudinal axis and a plurality of rings connected via connectors. Each ring is defined by a plurality of wave-like or undulating structures. Each undulating structure comprises a bar arm and crowns. Each bar arm has a first end portion, a second end portion opposing to the first end portion and a mid-section located between the first end portion and the second end portion. The bar arm has at least one perforated pattern on a surface thereof such that the first end portion is arranged with a perforated pattern on a surface thereof to be defined as a first opened region, the mid-section is arranged with a perforated pattern on a surface thereof to be defined as a second opened region, and the second end portion is arranged with a perforated pattern on a surface thereof to be defined as a third opened region. A surface area ratio between the surface area of the first opened region and the surface area of the first end portion is a first opening ratio, a surface area ratio between the surface area of the second opened region and the surface area of the mid-section is a second opening ratio, a surface area ratio between the surface area of the third opened region and the surface area of the second end portion is a third opening ratio, the second opening ratio is larger than the first opening ratio, and the second opening ratio is larger than the third opening ratio. Moreover, the crown is connected to the end portion of the bar arm.

The stent of the instant disclosure has a perforated pattern formed on the surface of the bar arm for drug retention. The perforated pattern provides higher structural strength in two end portions of the bar arm relatively to the mid-section of the bar arm. After the stent is implanted in blood vessels, the bar arm can share the loading that is originally concentrated on the crowns such that the overall stresses are more uniformly distributed across the stent as a whole. Thus, the fatigue life of the stent is enhanced.

In order to further understand the instant disclosure, the following embodiments and illustrations are provided. However, the detailed description and drawings are merely illustrative of the disclosure, rather than limiting the scope being defined by the appended claims and equivalents thereof.

### BRIEF DESCRIPTION OF THE DRAWINGS

FIG. 1A is a perspective view illustrating portions of a drug eluting depot stent in accordance with an embodiment of the instant disclosure;

FIG. 1B is a two-dimensional view of FIG. 1A illustrating the stent in accordance with the instant disclosure;

FIG. 1C is a cross-sectional view of FIG. 1B along the axis A-A in accordance with the instant disclosure;

FIG. 2 is a two-dimensional view of the drug eluting depot stent in accordance with another embodiment of the instant disclosure;

FIG. 3 is a two-dimensional view of the drug eluting depot stent in accordance with another embodiment of the instant disclosure;

FIG. 4 is a two-dimensional view of the drug eluting depot stent in accordance with another embodiment of the instant disclosure;

FIG. 5 is a two-dimensional view of the drug eluting depot stent in accordance with another embodiment of the instant disclosure;

FIG. 6 is a two-dimensional view of the drug eluting depot stent in accordance with another embodiment of the instant disclosure; and

FIG. 7 is a two-dimensional view of the drug eluting depot stent in accordance with another embodiment of the instant disclosure.

### DETAILED DESCRIPTION OF THE PREFERRED EMBODIMENTS

The aforementioned illustrations and detailed descriptions are exemplarities for the purpose of further explaining the scope of the instant disclosure. Other objectives and advantages related to the instant disclosure will be illustrated in the subsequent descriptions and appended drawings.

When a vascular stent is implanted into a blood vessel, the stent is positioned in a pre-determined location via a catheter. The diameter of the stent is expanded via balloon angioplasty or self-expanded via nitinol materials to dilate the narrowed segment of a blood vessel, such that preferred blood flow is restored there through. The instant embodiment furthers discloses the stent before expansion as follow.

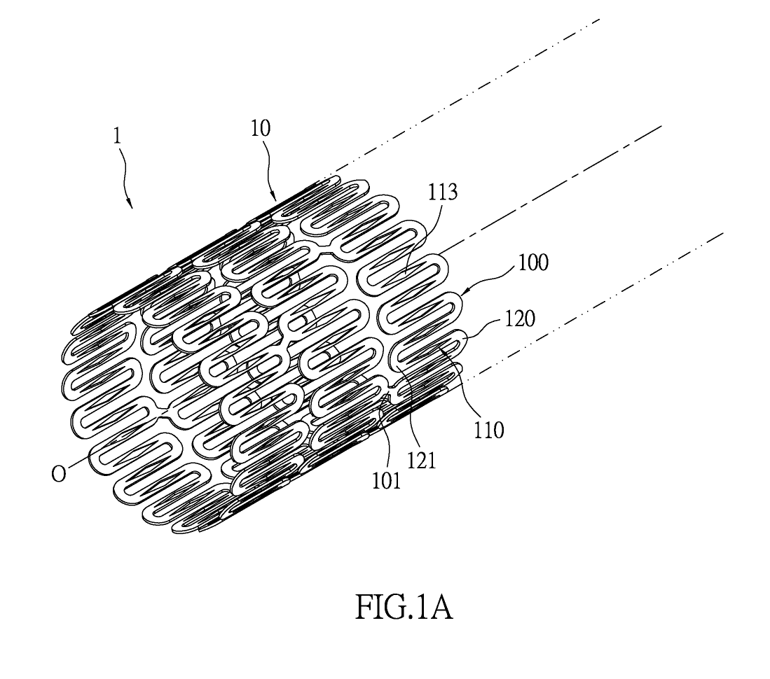

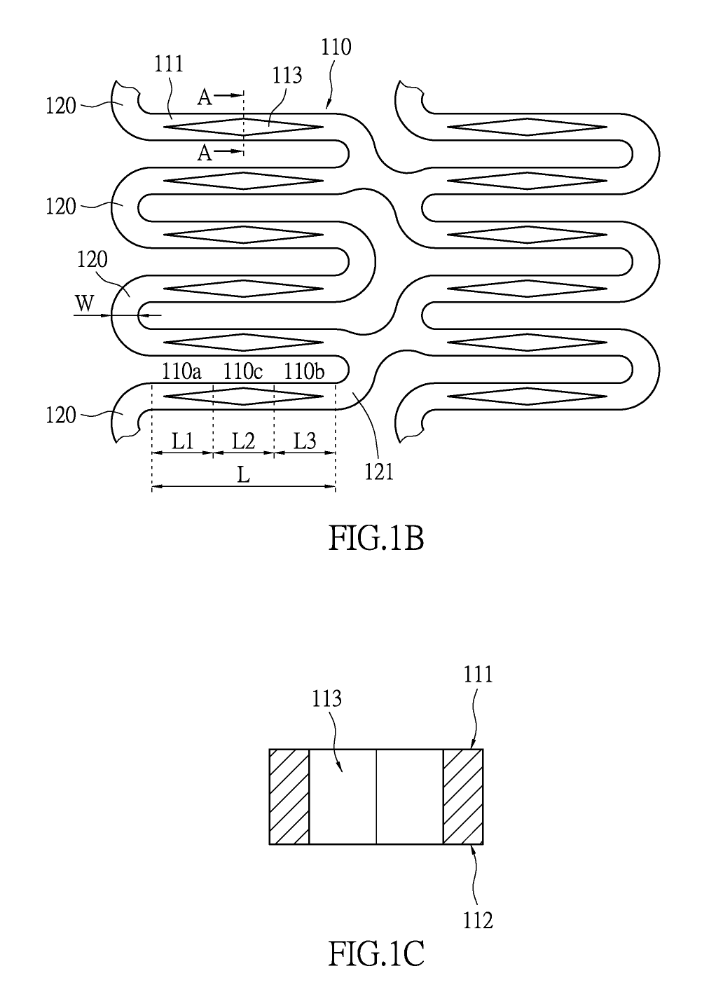

Please refer to FIG. 1A as the perspective view illustrating portions of a drug eluting depot stent in accordance with an embodiment of the instant disclosure. A stent 1 is made of metallic materials or bioabsorbable materials that are biocompatible such as stainless steel alloys, nickel titanium alloys, cobalt-chromium alloys, cobalt-nickel alloys, platinum chromium alloys, polylactic acids, L-lactic acids, polyglycolic acids, etc. The stent 1 has two free ends (not labeled) and a tubular body 10. The tubular body 10 extends throughout and in between the two free ends while having a longitudinal axis O. The tubular body 10 includes a plurality of rings 100. The rings 100 are connected in series along the longitudinal axis O to form the tubular body 10.

Specifically, any two adjacent rings 100 are connected via at least a connector 101. The connector 101 can be straight, curved, or any type of shape. In an embodiment, the rings 100 are formed by a plurality of wave-like or undulating structures. The undulating structure includes a bar arm 110, a first crown 120, and a second crown 121.

Please refer to FIG. 1B as a two-dimensional local view of FIG. 1A illustrating the stent in accordance with the instant disclosure. The bar arm 110 has a first end portion 110a, a second end portion 110b opposing to the first end portion 110a, and a mid-section 110c. The mid-section 110c is located between the first and the second end portions 110a, 110b. The first end portion 110a of the bar arm 110 is connected to the first crown 120, and the second end portion 110b is connected to the second crown 121. The first and the second crowns 120,121 may have different curvatures.

Please refer to FIGS. 1B and 1C. FIG. 1C is a cross-sectional view of FIG. 1B along the axis A-A in accordance with the instant disclosure. The bar arm 110 has a surface which includes an outer surface 111 and an inner surface 112 oppositely faced from the outer surface 111. In the instant embodiment, the outer wall surface of the tubular body 10 is made of the outer surfaces 111 of the bar arm 110, and the inner wall surface of the tubular body 10 is made of the inner surfaces 112 of the bar arm 110. After the stent 1 is implanted into blood vessels, the outer wall surface of the tubular body 10, or the outer surfaces 111 of the bar arm 110, is in close contact with the periphery of the inner walls of the blood vessels.

Please refer to FIG. 1B. In the instant embodiment, the bar arm 110 has a width that is substantially equivalent to a width W of the first crown 120 and also a width of the second crown 121. The width of bar arm 110 is substantially uniform from the first end portion 110a, across and to the second end portion 110b. However, the width of the bar arm 100 is not necessarily the same throughout the first end portion 110a and the second end portion 110b in another embodiment. The bar arm 110 has a perforated pattern arranged on a surface thereof. Specifically, the bar arm 110 has at least one opening formed on the outer surface 111 and/or the inner surface 112 to retain drugs therein. The perforated pattern formed on a surface of the first end portion 110a is defined as a first opened region, the perforated pattern formed on a surface of the mid-section 110c is defined as a second opened region, and the perforated pattern formed on a surface of the second end portion 110b is defined as a third opened region. The perforated pattern respectively arranged or formed on the first end portion 110a, the mid-section 110c, and the second end portion 110b can be the same or different from one another, thus the examples provided herein do not limit the shapes, sizes, or quantity of the perforated patterns.

The surface area of the first opened region on the first end portion 110a with respect to the surface area of the first end portion 110a is defined as a first opening ratio, the surface area of the second opened region on the mid-section 110c with respect to the surface area of the mid-section 110c is defined as a second opening ratio, and the surface area of the third opened region on the second end portion 110b with respect to the surface area of the second end portion 110b is defined as the third opening ratio. The second opening ratio is larger than the first opening ratio, and the second opening ratio is larger than the third opening ratio.

Specifically, the perforated pattern is a rhombic-shaped opened region 113 as shown in FIG. 1B. The rhombic-shaped opened region 113 is a rhombic opening in the instant embodiment. In other words, the bar arm 110 is formed with a rhombic-shaped opening, which can be a through hole or a blind hole, on the surface of the bar arm 110. In the instant embodiment, the rhombic opening is a through hole surrounded by the outer surface 111.

In the instant embodiment, the bar arm 110 is divided into three regions defined by the first end portion 110a, the mid-section 110c, and the second end portion 110b. In other words, the bar arm 110 has a length L substantially divided into three portions defined by the first end portion 110a, the mid-section 110c, and the second end portion 110b. The length L of the bar arm 110 is a distance between the two end portions thereof. In other words, the first end portion 110a has a length L1, the mid-section 110c has a length L2, and the second end portion 110b has a length L3. All the lengths L1, L2, L3 are substantially ⅓ of the length L.

In the instant embodiment, the rhombic opening extends from the first end portion 110a to the second end portion 110b, and the rhombic opening has a width at the center thereof which tapers towards the two end portions. In other words, the width of the rhombic opening enlarges from the first end portion 110a toward the mid-section 110c and reduces from the mid-section 110c to the second end portion 110b.

If the first end portion 110a has a surface area A1 (meaning W×L1), the mid-section 110c has a surface area A2 (W×L2), and the second end portion 110b has a surface area A3 (W×L3), whereas the first opened region formed by the rhombic opening 113 on the surface of the first end portion 110a has a surface area of B1, the second opened region on the surface of the mid-section 110c has a surface area of B2, and the third opened region on the surface of the second end portion 110b has a surface area of B3, then A1, A2, A3, B1, B2, and B3 satisfy the following conditions: (B2/A2)>(B1/A1), and (B2/A2)>(B3/A3). As such, the structural strength of the mid-section of each bar arm 110 is relatively weaker than the two end portions.

Specifically, when the stent 1 is implanted into the blood vessels, the stent 1 will radially support the wall of the blood vessels. In terms of the stent, the loading on the crown is typically higher than the loading on the bar arm. As a result, majority of the cracks initiates from the crown of the stent. Moreover, if the drug reservoir or depot on the bar arm has a uniform width, the reservoir or depot can significantly reduce the average service life of the stent with respect to the stent without drug reservoirs.

However, the stent 1 in the instant embodiment has the second opening ratio of the mid-section 110c larger than the first opening ratio of the first end portion 110a and the third opening ratio of the second end portion 110b via variations of the perforated pattern. As such, the structure of the bar arm 110 gradually weakens from two end portions towards the center thereof, and the stress concentration on the crown 120 can be guided towards the bar arm 110, such that fatigue life of the stent is increased. Although the stent 1 of the instant embodiment has the perforated pattern for drug retention, its fatigue life does not reduce but in contrast increases.

As shown in FIG. 1C, the rhombic opening 113 can penetrate through the outer surface 111 to the inner surface 112. When the rhombic opening 113 is a blind hole, the opening 113 can be formed on the outer surface 111 and/or the inner surface 112. The opening 113 can be used to retain one or more types of drugs therein. In an embodiment, when the rhombic opening 113 is a through hole, the upper and lower half of the opening 113 can be used to retain different types of drugs.

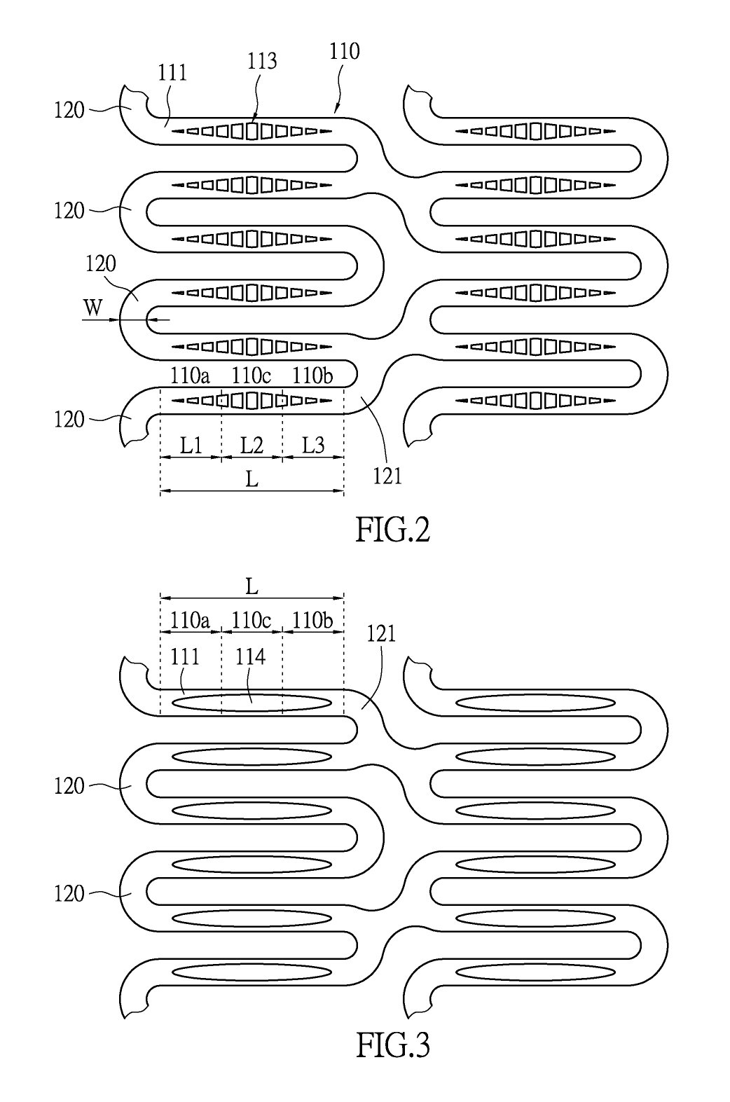

FIG. 2 is a two-dimensional view of the drug eluting depot stent in accordance with another embodiment of the instant disclosure. In another embodiment, a plurality of discontinuous openings of various sizes and shapes are arranged in such way that the overall shape of the openings resembles the contour of a rhombus. In other words, the perforated pattern includes a plurality of openings of various sizes and shapes that are not interconnected to each other.

FIG. 3 is a two-dimensional view of the drug eluting depot stent in accordance with another embodiment of the instant disclosure. In the instant embodiment, the perforated pattern is an elliptical opening 114. The elliptical opening 114 has a longitudinal axis which aligns with the first end portion 110a and the second end portion 110b. The length of the longitudinal axis of the opening 114 can be less than the length L of the bar arm 110, but is not limited herein.

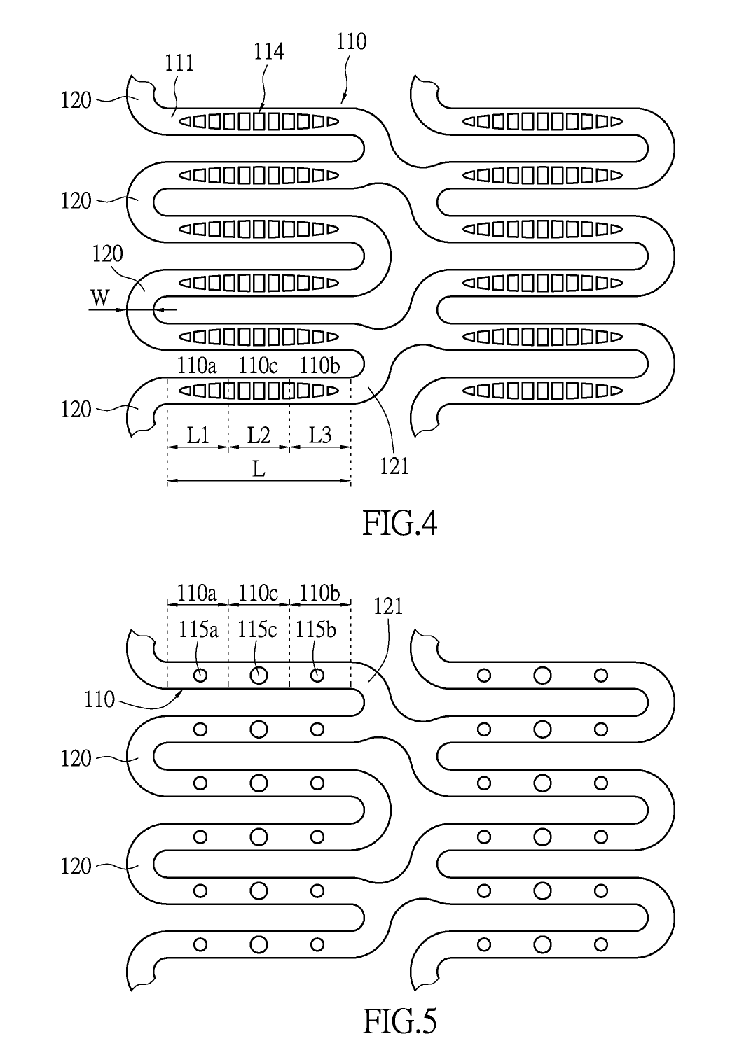

Please refer to FIG. 4 as a two-dimensional view of the drug eluting depot stent in accordance with another embodiment of the instant disclosure. In the instant embodiment, a plurality of discontinuous or non-interconnected openings having various sizes and shapes are arranged in such a way that the overall shape of the opening 114 resembles an ellipse. In other embodiment, the perforated pattern can also have a polygonal shape. The perforated pattern takes up a certain surface area on the outer surface 111 of the bar arm 110, and the occupied surface area ratio of the perforated pattern at the mid-section 110c is respectively larger than the occupied surface area ratios of the perforated patterns at the two end portions.

In another embodiment, the bar arm 110 has a plurality of tiny holes arranged on a surface thereof to form the perforated pattern. Please refer to FIG. 5 as a two-dimensional view of the drug eluting depot stent in accordance with another embodiment of the instant disclosure. In the instant embodiment, the perforated pattern includes a plurality of holes for example three holes 115a, 115b, 115c, as shown in FIG. 5. Specifically, the bar arm 110 has the plurality of holes 115a-115c, where the hole 115a is arranged on the surface of the first end portion 110a, the hole 115c is arranged on the surface of the mid-section 110c, and the hole 115b is arranged on the surface of the second end portion 110b. In the instant embodiment, the first end portion 110a, the mid-section 110c, and the second end portion 110b each has one hole 115, however, the number of holes 115 on the first end portion 110a, the mid-section 110c, and the second end portion 110b is not limited to the example provided herein.

In the instant embodiment, the width of the bar arm 110 across the first end portion 110a and the second end portion 110b are substantially uniform, however, the hole 115c has diameter respectively larger than the diameters of the holes 115a, 115b. In other words, the two dimensional surface area of the outer surface 111 of the mid-section 110c occupied by the hole 115c is larger than that of the first end portion 110a occupied by the hole 115a, and the two dimensional surface area of the outer surface 111 of the mid-section 110c which is occupied by the hole 115c is also larger than that of the second end portion 110b which is occupied by the hole 115b. As a result, due to the distribution of the holes 115a-115c, the structural strengths at two end portions (first and second end portions 110a, 110b) are stronger than that at the mid-section 110c. The cross-sectional shapes of the holes 115a-115c are circles as shown in FIG. 5, but they can also be polygons, ellipses or other shapes in other embodiments. The two dimensional surface areas of the first end portion 110a, the mid-section 110c, and the second end portion 110b respectively occupied by the holes 115a-115c are not limited to only the outer surface 111, and may also refer to the inner surface 112.

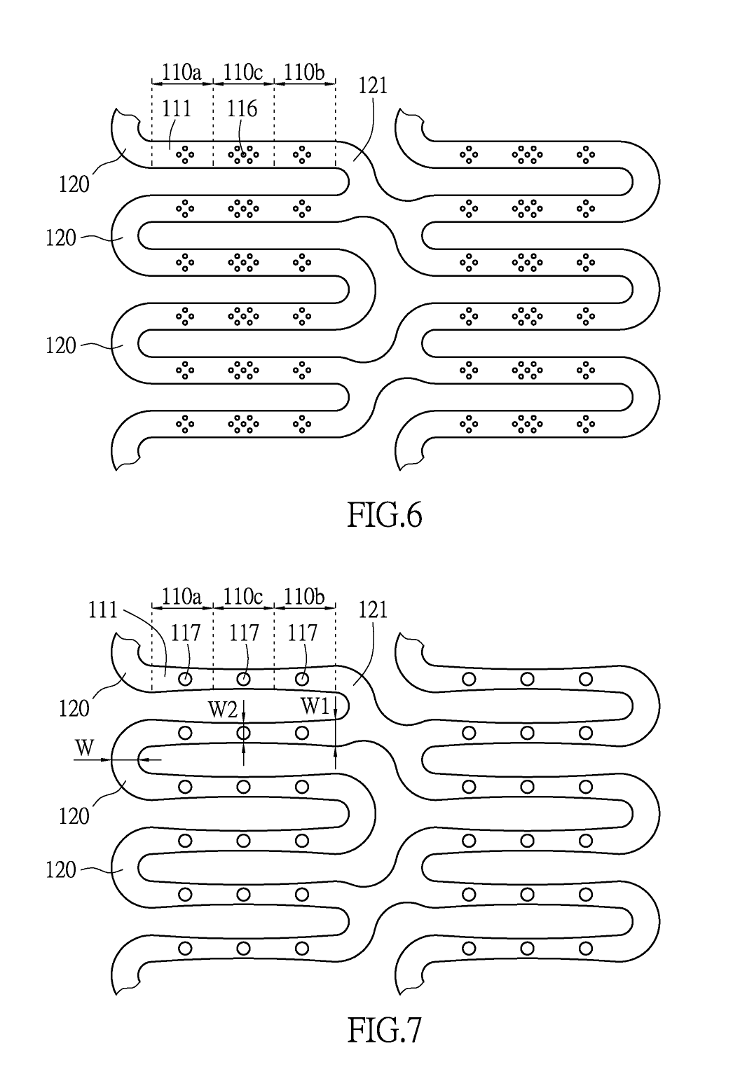

FIG. 6 is a two-dimensional view of the drug eluting depot stent in accordance with another embodiment of the instant disclosure. In the instant embodiment, the perforated pattern can have a plurality of discontinuous or non-interconnected tiny holes 116. Specifically, the bar arm 110 has a plurality of holes 116 distributed across the outer surface 111. The holes 116 have substantially similar diameters and have a larger distribution across the mid-section 110c than the two end portions (first and second end portions 110a, 110b). Moreover, the distribution of holes 116 is substantially similar across the two end portions 110a, 110b.

Furthermore, the width of the bar arm 110 may vary, yet the second opening ratio of the mid-section 110c is still respectively larger than the opening ratios of the first and the second end portions 110a, 110b. Please refer to FIG. 7 as a two-dimensional view of the drug eluting depot stent in accordance with another embodiment of the instant disclosure. In the instant embodiment, the bar arm 110 has a first width W1 at the first and the second end portions 110a, 110b, and a second width W2 at the mid-section 110c. The first width W1 is substantially similar to the width W of the first crown 120, and the second width W2 is smaller than the first width W1. In other words, the width of the bar arm 110 tapers from two end portions (first and second end portions 110a, 110b) towards the mid-section 110c.

Moreover, in the instant embodiment, the perforated pattern also has a plurality of discontinuous or non-interconnected tiny holes 117 (as shown in FIG. 7) distributed on the outer surface 111 of the bar arm 110. The holes 117 have substantially similar diameters. In another embodiment, the holes 117 may have different diameters. In addition, the holes 117 are distributed along the first end portion 110a, the mid-section 110c, and the second end portion 110b. In the instant embodiment, the first end portion 110a, the mid-section 110c, and the second end portion 110b each has one hole 117, however, the number of holes 117 on the first end portion 110a, the mid-section 110c, and the second end portion 110b is not limited to the example provided herein.

In the instant embodiment, the second width W2 of the bar arm 110 is smaller than the first width W1 such that the surface area A2 of the mid-section 110c is respectively smaller than the surface area A1 of the first end portion 110a, and the surface area A3 of the second end portion 110b. Although the holes 117 are distributed at the first end portion 110a, the mid-section 110c, and the second end portion 110b, the diameters of the holes are substantially similar, however, the second opening ratio (B2/A2) are still the largest among all.

The perforated patterns on the outer surface 111 of the bar arm 110 in aforementioned embodiments are only exemplary and does not limit to the examples provided herein. The perforated pattern can also be formed on the inner surface 112 of the bar arm 110. Alternatively, the perforated patterns can be blind holes separately formed on the outer surface 111 and the inner surface 112.

As mentioned in previous embodiments, the first crown 120 and the second crown 121 usually do not have any openings on their respective surfaces. After the stent 1 radially expands, the first and second crowns 120, 121 typically have the maximum stresses on them. If the first and second crowns 120, 121 have the perforated pattern on their respective surfaces, the structure of the first and second crowns 120, 121 can be weakened such that the service life of the stent is substantially reduced.

In summary, the stent provided in accordance with the embodiments of the instant disclosure has at least an opening on the bar arm for drug retention. The structural strength of the bar arm gradually increases from the mid-section towards the two end portions due to the presence of one opening or multiple openings. As a result, when the stent is implanted into blood vessels, the bar arm shares more loadings with the crowns when the stent is radially expanded. In comparison to conventional stents, the stent of the instant disclosure has more uniform load distribution across its entirety, which enhances the fatigue life of the stent. Moreover, with the openings for drug retention on the bar arm of the stent, the service life of the stent is not reduced, but in contrast, is increased.

The figures and descriptions supra set forth illustrated the preferred embodiments of the instant disclosure; however, the characteristics of the instant disclosure are by no means restricted thereto. All changes, alternations, combinations or modifications conveniently considered by those skilled in the art are deemed to be encompassed within the scope of the instant disclosure delineated by the following claims.

What is claimed is:

1. A drug eluting depot stent having two free ends and a tubular body, the tubular body having a longitudinal axis and including a plurality of rings, the rings are connected along the longitudinal axis via connectors, each ring is defined by a plurality of undulating structures, each undulating structure comprising:
a bar arm having a first end portion, a second end portion opposing to the first end portion and a mid-section, the bar arm having at least one perforated pattern on a surface thereof, the first end portion with a perforated pattern on a surface thereof defined as a first opened region, the mid-section of the bar arm with a perforated pattern on a surface thereof defined as a second opened region, the second end portion with a perforated pattern on a surface thereof defined as a third opened region; and
a first crown connected to the first end portion of the bar arm;
wherein the mid-section is located between the first end portion and the second end portion, a surface area ratio between the surface area of the first opened region and the surface area of the first end portion is a first opening ratio, a surface area ratio between the surface area of the second opened region and the surface area of the mid-section is a second opening ratio, a surface area ratio between the surface area of the third opened region and the surface area of the second end portion is a third opening ratio, the second opening ratio is larger than the first opening ratio, and the second opening ratio is larger than the third opening ratio.

2. The drug eluting depot stent as recited in claim 1, wherein the bar arm has a substantially uniform width across the first end portion to the second end portion.

3. The drug eluting depot stent as recited in claim 2, wherein the bar arm has an opening on the surface thereof to form the perforated pattern, the opening is across the first end portion and the second end portion, and the opening has a width thereof tapering from the mid-section towards two end potions.

4. The drug eluting depot stent as recited in claim 3, wherein the opening is a rhombic-shaped opening, an elliptical-shaped opening, or a polygonal-shaped opening.

5. The drug eluting depot stent as recited in claim 3, wherein the opening is a through hole or a blind hole.

6. The drug eluting depot stent as recited in claim 2, wherein the surface of the first end portion, the surface of the second end portion, and the surface of the mid-section respectively have at least one hole arranged thereon to form the perforated pattern, and the holes of the mid-section have the largest diameter among all.

7. The drug eluting depot stent as recited in claim 2, wherein the bar arm has a plurality of holes arranged on the surface thereof to form the perforated pattern.

8. The drug eluting depot stent as recited in claim 7, wherein the mid-section of the bar arm has the maximum amount of holes arranged thereon.

9. The drug eluting depot stent as recited in claim 1, wherein the first end portion and the second end portion of each of the bar arms have a first width, the mid-section of each bar arm has a second width, and the second width is smaller than the first width.

10. The drug eluting depot stent as recited in claim 9, wherein the surface of the first end portion, the surface of the second end portion, and the surface of the mid-section respectively have at least one hole arranged thereon to form the perforated pattern, and the holes of the first end portion, second end portion, and the mid-section have similar diameters.

11. The drug eluting depot stent as recited in claim 1, wherein the bar arm has a surface including an inner surface and an outer surface opposing to the inner surface, the perforated pattern is arranged on the inner surface or the outer surface.

12. The drug eluting depot stent as recited in claim 1, wherein the undulating structure further comprising:
a second crown connected to the second end portion of the bar arm.

13. The drug eluting depot stent as recited in claim 1, wherein the perforated pattern defined as an opened region, the opened region has a plurality of discontinuous openings.

14. The drug eluting depot stent as recited in claim 13, wherein the plurality of openings jointly resembles a rhombic, an elliptical, or a polygonal shape.
